(19) United States
(12) Patent Application Publication
(10) Pub. No.: US 2026/0013569 A1
(43) Pub. Date: Jan. 15, 2026

(54) INTELLIGENT ELECTRONIC CIGARETTE WITH MONITORING FUNCTION

(71) Applicant: Shenzhen Xingaodeng Technology Co., Ltd, Shenzhen (CN)

(72) Inventor: Meiqi Wang, Shenzhen (CN)

(21) Appl. No.: 18/769,316

(22) Filed: Jul. 10, 2024

(51) Int. Cl. A24F 40/53; A24F 40/10; A24F 40/40; A24F 40/51; A24F 40/60; A24F 40/65; A24F 40/90; A61B 5/00; A61B 5/0205

(52) U.S. Cl. CPC A24F 40/53; A24F 40/10; A24F 40/40; A24F 40/51; A24F 40/60; A24F 40/65; A24F 40/90; A61B 5/0205; A61B 5/6887

(57) ABSTRACT

The present disclosure relates to the technical field of electronic cigarettes, and in particular to an intelligent electronic cigarette with a monitoring function, including an electronic cigarette body, a cigarette holder, a host integration module, a sensor, a radio frequency identification (RFID) magnetic code identification module, a gas monitoring module and a Bluetooth and wireless fidelity (Wi-Fi) module. The host integration module, the sensor, the RFID magnetic code identification module, the gas monitoring module and the Bluetooth and Wi-Fi module are located at an interior of the electronic cigarette body. By integrating multiple intelligent monitoring and health monitoring functions, as well as intelligent identification technology, users can be provided with the safer, convenient and personalized use experience of the electronic cigarette. Meanwhile, through the Bluetooth and Wi-Fi module, user's health reports can be easily stored, printed or uploaded to hospitals or private physicians, providing strong support for user's health management.

### TECHNICAL FIELD

The present disclosure relates to the technical field of electronic cigarettes, and in particular to an intelligent electronic cigarette with a monitoring function.

### BACKGROUND

In recent years, with the continuous expansion of the electronic cigarette market and the improvement of consumer health awareness, the requirements for the safety and functionality of electronic cigarette products are increasing. Although traditional electronic cigarettes can simulate the smoking experience of traditional tobacco, during use, the lack of effective monitoring and feedback mechanisms cannot ensure the health and safety of users.

At present, most of the electronic cigarette products on the market focus only on the taste and aroma of smoke, but neglect the impact of possible harmful substances on the health of users during the use of electronic cigarette. For example, carbon monoxide and carbon dioxide are common harmful substances in electronic cigarette smoke, and the long-term inhalation of electronic cigarette smoke may cause damage to the user's respiratory system. In addition, existing electronic cigarette products often fail to provide personalized adjustment options because different users have different preferences for the concentration and temperature of electronic cigarette smoke, making the users unable to acquire the best smoking experience.

With the rapid development of intelligent technology, combining monitoring technology with electronic cigarette products can provide the users with a safer, healthier and more intelligent smoking experience. By integrating the sensor and the monitoring module, the intelligent electronic cigarette can monitor the content of harmful substances in smoke in real time, smoke concentration, temperature and other key parameters, and display the monitored data to the user through the display screen or mobile phone application, so that the user can understand the health status and smoke state in a process of smoking.

In addition, the intelligent electronic cigarette can also integrate the health monitoring functions, such as heart rate monitoring and oxygen saturation monitoring, to provide the users with immediate health feedback through the real-time monitoring of user's physiological indicators. When the user's body is abnormal, the intelligent electronic cigarette can automatically remind the user and take corresponding measures, such as reducing the smoke concentration or turning off the power supply, to ensure the user's health and safety.

Therefore, the present disclosure provides an intelligent electronic cigarette with a monitoring function, aiming to provide a user with a safer, healthier and more intelligent smoking experience by integrating a monitoring technology and an intelligent control function. The intelligent electronic cigarette can not only monitor the content of harmful substances in smoke and the smoke status in real time, but also integrate the function of health monitoring to provide the users with immediate health feedback and personalized adjustment options to meet the needs of different users.

### SUMMARY

An object of the present disclosure is to provide an intelligent electronic cigarette with a monitoring function to solve the problems set forth in the above background.

In order to achieve the above object, the present disclosure provides the following technical solutions.

In an alternative solution of the intelligent electronic cigarette with a monitoring function according to the present disclosure: an intelligent electronic cigarette with a monitoring function includes an electronic cigarette body, a cigarette holder, a host integration module, a sensor, a radio frequency identification (RFID) magnetic code identification module, a gas monitoring module and a Bluetooth and wireless fidelity (Wi-Fi) module. The host integration module, the sensor, the RFID magnetic code identification module, the gas monitoring module and the Bluetooth and Wi-Fi module are located at an interior of the electronic cigarette body;
the cigarette holder is mounted at a top of the electronic cigarette body;
a switch button is mounted on a side surface of the electronic cigarette body, and a hanging ring fixedly connected to the electronic cigarette body is arranged above the switch button; and
a display screen is further mounted on a surface of the electronic cigarette body.

Preferably, to further optimize the above technical solution, a charging port for connecting a charging line is further disposed on a side surface of the electronic cigarette body.

Preferably, to further optimize the above technical solution, the cigarette holder is an electronic cigarette cartridge or a cannabidiol (CBD) suction nozzle; and the sensor and the RFID magnetic code identification module mounted are configured to automatically identify liquid content, compositions, a manufacturer and origin information of the cigarette cartridge.

Preferably, to further optimize the above technical solution, the interior of the electronic cigarette body further includes a carbon monoxide and carbon dioxide content monitoring module, and an atomizing gas concentration and temperature adjustment and monitoring module for monitoring and adjusting gas compositions and a temperature generated by the electronic cigarette.

Preferably, to further optimize the above technical solution, the host integration module includes a body temperature, heart rate, oxygen saturation, blood pressure and electrocardiogram health monitoring module for monitoring health conditions of a user in real time.

Preferably, to further optimize the above technical solution, the Bluetooth and Wi-Fi module is configured to store, print or upload monitored health reports to hospitals or private physicians.

Preferably, to further optimize the above technical solution, the host integration module has the functions of health abnormality reminding and automatic power-off; and when the health abnormality of the user is detected, a power is automatically turned off to ensure the safety of the user.

Preferably, to further optimize the above technical solution, the electronic cigarette body further includes a post-atomizing gas monitoring module for monitoring the number of remaining smoking times and displaying the same on the display screen as well as a remaining amount of the cigarette cartridge and various taste information.

Preferably, to further optimize the above technical solution, a smoke concentration may be set as strong, medium and weak, and relevant information is displayed on the screen as well as the manufacturer, product detailed parameters and hemp producing area information.

Preferably, to further optimize the above technical solution, the interior of the electronic cigarette body further includes a harmful substance monitoring module; and when the module detects that the displayed harmful substance exceeds the standard, information is displayed on the display screen.

Compared with the prior art, the present disclosure has the following advantageous effects.

When the intelligent electronic cigarette is used, by integrating multiple intelligent monitoring and health monitoring functions, as well as intelligent identification technology, users can be provided with the safer, convenient and personalized use experience of the electronic cigarette. Meanwhile, through the Bluetooth and Wi-Fi module, user's health reports can be easily stored, printed or uploaded to hospitals or private physicians, providing strong support for user's health management.

### BRIEF DESCRIPTION OF THE DRAWINGS

FIG. 1 is a schematic diagram of an overall structure of the present disclosure.

Reference numerals and denotations thereof: 1—electronic cigarette body; 2—cigarette holder; 3—hanging ring; 4—switch button; and 5—display screen.

### DETAILED DESCRIPTION

Technical solutions in examples of the present disclosure will be described clearly and completely in the following with reference to the attached drawings in the examples of the present disclosure. Obviously, all the described examples are only some, rather than all examples of the present disclosure. Based on the examples in the present disclosure, all other examples obtained by those of ordinary skill in the art without creative efforts belong to the scope of protection of the present disclosure.

### Example 1

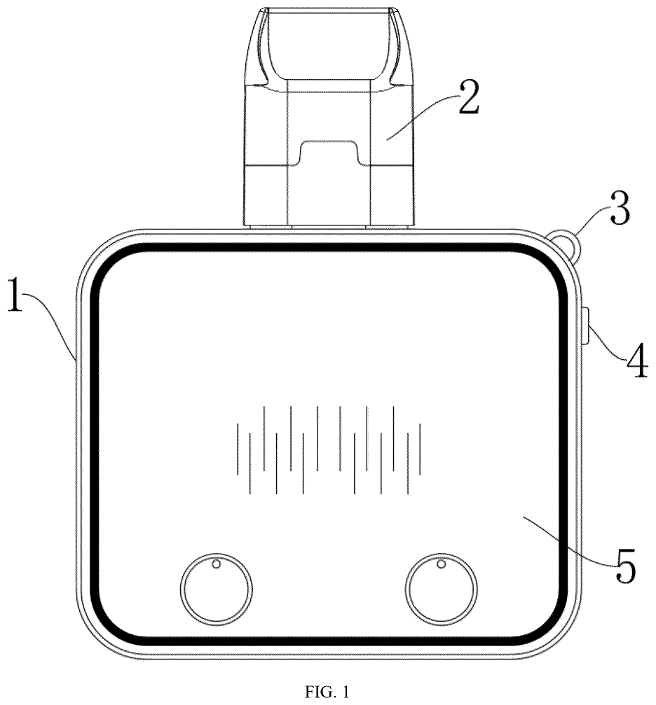

Referring to FIG. 1, the present disclosure provides the following technical solution:

An intelligent electronic cigarette with a monitoring function includes an electronic cigarette body 1, a cigarette holder 2, a host integration module, a sensor, an RFID magnetic code identification module, a gas monitoring module and a Bluetooth and Wi-Fi module. The host integration module, the sensor, the RFID magnetic code identification module, the gas monitoring module and the Bluetooth and Wi-Fi module are located at an interior of the electronic cigarette body 1;
the cigarette holder 2 is mounted at a top of the electronic cigarette body 1;
a switch button 4 is mounted on a side surface of the electronic cigarette body 1, and a hanging ring 3 fixedly connected to the electronic cigarette body 1 is arranged above the switch button 4; and
a display screen 5 is further mounted on a surface of the electronic cigarette body 1.

To further optimize the above technical solution, a charging port for connecting a charging line is further disposed on a side surface of the electronic cigarette body 1.

To further optimize the above technical solution, the cigarette holder 2 is an electronic cigarette cartridge or a CBD suction nozzle; and the sensor and the RFID magnetic code identification module mounted are configured to automatically identify liquid content, compositions, a manufacturer and origin information of the cigarette cartridge.

To further optimize the above technical solution, the interior of the electronic cigarette body 1 further includes a carbon monoxide and carbon dioxide content monitoring module, and an atomizing gas concentration and temperature adjustment and monitoring module for monitoring and adjusting gas compositions and a temperature generated by the electronic cigarette.

To further optimize the above technical solution, the host integration module includes a body temperature, heart rate, oxygen saturation, blood pressure and electrocardiogram health monitoring module for monitoring health conditions of a user in real time.

To further optimize the above technical solution, the Bluetooth and Wi-Fi module is configured to store, print or upload monitored health reports to hospitals or private physicians.

To further optimize the above technical solution, the host integration module has the functions of health abnormality reminding and automatic power-off; and when the health abnormality of the user is detected, a power is automatically turned off to ensure the safety of the user.

To further optimize the above technical solution, the electronic cigarette body 1 further includes a post-atomizing gas monitoring module for monitoring the number of remaining smoking times and displaying the same on the display screen as well as a remaining amount of the cigarette cartridge and various taste information.

To further optimize the above technical solution, a smoke concentration may be set as strong, medium and weak, and relevant information is displayed on the screen as well as the manufacturer, product detailed parameters and hemp producing area information.

To further optimize the above technical solution, the interior of the electronic cigarette body further includes a harmful substance monitoring module; and when the module detects that the displayed harmful substance exceeds the standard, information is displayed on the display screen.

Compared to conventional electronic cigarette products, the intelligent electronic cigarette with a monitoring function according to the present disclosure has the following significant beneficial effects.

Health and safety assurance: in the present disclosure, by integrating the carbon monoxide and carbon dioxide content monitoring module and the atomizing gas concentration and temperature adjusting and monitoring module, the gas compositions and temperature generated by the electronic cigarette can be monitored in real time, ensuring that the smoke inhaled by the user meets safety standards and greatly reducing the potential health risks associated with the use of the electronic cigarette.

Personalized adjustment: a smoke concentration can be set as strong, medium and weak to meet the personalized needs of different users. Meanwhile, key parameters such as smoke concentration and temperature can be displayed on the display screen, and the user can make real-time adjustment according to requirements, to acquire the best smoking experience.

Intelligent monitoring and reminding: the body temperature, heart rate, oxygen saturation, blood pressure and electrocardiogram health monitoring apparatus contained in the host integration module can monitor the health conditions of the user in real time. When it is detected that the user's health is abnormal, the electronic cigarette can automatically remind the user and take corresponding measures, such as reducing the smoke concentration or turning off the power to ensure the safety of the user.

Information traceability and anti-counterfeiting: in the present disclosure, through the RFID magnetic code identification apparatus, the liquid content, compositions, manufacturer and origin information of the cigarette cartridge can be automatically identified, realizing the automatic identification and tracing of product information, which not only helps the user to understand the detailed information of the product, but also improves the anti-counterfeiting ability of the product and guarantees the user's rights and interests.

Intelligent management and service: the integration of the Bluetooth and Wi-Fi module enables the intelligent electronic cigarette to store, print, or upload the monitored health reports to hospitals or private physicians, facilitating the users to view and manage health data at any time. Meanwhile, it also provides the hospitals or private physicians with more comprehensive user health data and helps to provide more accurate health services.

Convenience and practicality: the design of the hanging ring is convenient for the user to carry and hang the electronic cigarette; the arrangement of the charging port makes the charging of the electronic cigarette more convenient; the real-time display function of the display screen enables the user to understand the working state and health state of the electronic cigarette at any time.

In summary, the intelligent electronic cigarette with a monitoring function according to the present disclosure not only improves the safety and health of an electronic cigarette product, but also provides the more personalized and intelligent smoking experience and management service for the user, with significant beneficial effects.

It is to be noted that in this specification, relational terms such as “first” and “second” are only used to distinguish one entity or operation from another entity or operation, and do not necessarily require or imply that there is any such an actual relationship or order between these entities or operations. Moreover, the terms “including”, “containing” or any other variations are intended to cover non-exclusive inclusion, so that a process, method, article or device including a series of elements includes not only those elements, but also other elements not explicitly listed or elements inherent to such a process, method, article or device.

While examples of the present disclosure have been shown and described, it will be understood by those skilled in the art that various changes, modifications, substitutions and alterations may be made herein without departing from the principles and spirit of the present disclosure, the scope of which is defined by the appended claims and equivalents thereof.

1. An intelligent electronic cigarette with a monitoring function, comprising:
an electronic cigarette body (1), a cigarette holder (2), a host integration module, a sensor, a radio frequency identification (RFID) magnetic code identification module, a gas monitoring module and a Bluetooth and wireless fidelity (Wi-Fi) module, wherein the host integration module, the sensor, the RFID magnetic code identification module, the gas monitoring module and the Bluetooth and Wi-Fi module are located at an interior of the electronic cigarette body (1);
the cigarette holder (2) is mounted at a top of the electronic cigarette body (1);
a switch button (4) is mounted on a side surface of the electronic cigarette body (1), and a hanging ring (3) fixedly connected to the electronic cigarette body (1) is arranged above the switch button (4); and
a display screen (5) is further mounted on a surface of the electronic cigarette body (1).

2. The intelligent electronic cigarette with a monitoring function according to claim 1, wherein a charging port for connecting a charging line is further disposed on a side surface of the electronic cigarette body (1).

3. The intelligent electronic cigarette with a monitoring function according to claim 1, wherein the cigarette holder (2) is an electronic cigarette cartridge or a cannabidiol (CBD) suction nozzle; and the sensor and the RFID magnetic code identification module mounted are configured to automatically identify liquid content, compositions, a manufacturer and origin information of the cigarette cartridge.

4. The intelligent electronic cigarette with a monitoring function according to claim 3, wherein the interior of the electronic cigarette body (1) further comprises a carbon monoxide and carbon dioxide content monitoring module, and an atomizing gas concentration and temperature adjustment and monitoring module for monitoring and adjusting gas compositions and a temperature generated by the electronic cigarette.

5. The intelligent electronic cigarette with a monitoring function according to claim 4, wherein the host integration module comprises a body temperature, heart rate, oxygen saturation, blood pressure and electrocardiogram health monitoring module for monitoring health conditions of a user in real time.

6. The intelligent electronic cigarette with a monitoring function according to claim 5, wherein the Bluetooth and Wi-Fi module is configured to store, print or upload monitored health reports to hospitals or private physicians.

7. The intelligent electronic cigarette with a monitoring function according to claim 6, wherein the host integration module has the functions of health abnormality reminding and automatic power-off; and when the health abnormality of the user is detected, a power is automatically turned off to ensure the safety of the user.

8. The intelligent electronic cigarette with a monitoring function according to claim 7, wherein the electronic cigarette body (1) further comprises a post-atomizing gas monitoring module for monitoring the number of remaining smoking times and displaying the same on the display screen as well as a remaining amount of the cigarette cartridge and various taste information.

9. The intelligent electronic cigarette with a monitoring function according to claim 8, wherein a smoke concentration may be set as strong, medium and weak, and relevant information is displayed on the screen as well as the manufacturer, product detailed parameters and hemp producing area information.

10. The intelligent electronic cigarette with a monitoring function according to claim 1, wherein the interior of the electronic cigarette body (1) further comprises a harmful substance monitoring module; and when the module detects that the displayed harmful substance exceeds the standard, information is displayed on the display screen (5).
